(19) United States
(12) Patent Application Publication
(10) Pub. No.: US 2021/0335494 A1
(43) Pub. Date: Oct. 28, 2021

(54) Digital Contact Tracing Through Virtual Medical Information Portfolio and On-Ramp to Testing

(71) Applicant: Stanley Victor CAMPBELL, Vienna, VA (US)

(72) Inventor: Stanley Victor CAMPBELL, Vienna, VA (US)

(21) Appl. No.: 17/241,416

(22) Filed: Apr. 27, 2021

Related U.S. Application Data
(60) Provisional application No. 63/016,274, filed on Apr. 27, 2020.

(51) Int. Cl. G16H 50/30; A61B 5/0205

(52) U.S. Cl. CPC G16H 50/30; A61B 5/0205

(57) ABSTRACT

The digital contact tracing through virtual medical information portfolio and on-ramp to testing includes systems, devices, and computer-implemented methods for monitoring health of a user. The user receives a text message containing a QR code that directs the user to a patient survey. The user is categorized into one of a plurality of categories based on user data input into the patient survey. Subsequently, the user inputs one or more physiological parameters that allow remote monitoring of the patient. The one or more physiological parameters are compared against respective thresholds and change thresholds.

### PRIORITY

This application claims the benefit of U.S. Patent Application No. 63/016,274 filed on Apr. 27, 2020, which is hereby incorporated herein by reference in its entirety.

### FIELD OF THE INVENTION

The present invention generally relates to health care systems, and more importantly, to healthcare systems that provide pandemic control through contact tracing.

### BACKGROUND OF THE INVENTION

In recently published studies of COVID-19 patients in Wuhan, China, the initial indicators of infection were elevated temperature, followed by increased heart rate and respiratory rate, along with a decrease in oxygen saturation levels along with other clinical symptoms. In the absence of herd immunity, widespread vaccination, and/or effective treatments that prevent the acquisition of an infectious disease, a critical component of reducing disease transmission is surveilling contacts of confirmed cases and tracking them and their contacts as early as possible. The early recognition of the changes in clinical presentation can identify potential cases warranting further tiered diagnostic testing, and appropriate interventions.

The current literature and reports also confirm that many individuals with flu-like symptoms in fact test negative, while those with positive tests may be completely asymptomatic. Further, there is a variable length of viral shedding based on post-quarantine or post-treatment testing where COVID-19 tests remain positive. Thus, a single COVID-19 test result should not alone drive decision-making or behavior change, but rather that other available data elements should be collected, aggregated, and analyzed to inform policy. When individuals who have had contact with a confirmed case are identified, such individuals can be monitored, and promptly isolated if they also develop symptoms of the disease, thereby preventing further transmission of the disease. Finding and isolating infected people before they transmit further allows epidemiologists to break chains of transmission.

Globally, characterization of the threat of COVID-19 is severely limited by the global disparities in critical infrastructure and the availability of testing to define the actual incidence of the disease, among other factors. Recognized as a worldwide pandemic, COVID-19 does not as of yet have a cure and the current treatments will require an undetermined number of additional dosing, thus monitoring and containment of the disease remains among the most effective courses of action. It also remains uncertain as to whether currently available vaccines will remain effective against future variants. Nationwide efforts to mitigate the outbreak have been met by communication and technological setbacks, including inconsistent testing methods or lack of availability of testing altogether.

Epidemiologists need a way to identify contacts who may have become infected and/or vaccinated and monitor them for disease development. The tool that epidemiologists typically use is an in-person or telephone-based interview of an initial confirmed case. However, as the number of infected has grown dramatically on a national and international scale whether the person has been vaccinated or not, the health care system evidences significant stress, and control of COVID-19 requires automated systems and methods that are designed to grow to large-scale.

### SUMMARY OF THE INVENTION

Accordingly, the present invention is directed to digital contact tracing through virtual medical information portfolio and on-ramp to testing that substantially obviates one or more problems due to limitations and disadvantages of the related art.

Additional features and advantages of the invention will be set forth in the description which follows, and in part will be apparent from the description, or may be learned by practice of the invention. The objectives and other advantages of the invention will be realized and attained by the structure particularly pointed out in the written description and claims hereof as well as the appended drawings.

To achieve these and other advantages and in accordance with the purpose of the present invention, as embodied and broadly described, the digital contact tracing through virtual medical information portfolio and on-ramp to testing includes a computer-implemented method for monitoring health of a user, the method comprising receiving a text or other electronic message containing a QR code, bar code or other readable identifier that directs the user to a patient survey, categorizing the user into one of a plurality of categories based on user data input into the patient survey, inputting, by the user, one or more physiological or clinical parameters that allow remote monitoring of the patient, and comparing each of the one or more physiological parameters against a respective threshold and a respective change threshold.

In another aspect, the digital contact tracing through virtual medical information portfolio and on-ramp to testing includes a portable electronic device for monitoring health of a user, the portable electronic device comprising a processor, a storage memory coupled to the processor, the processor configured to receiving a text or other electronic message containing a QR code, bar code or other readable identifier that directs the user to a patient survey, categorizing the user into one of a plurality of categories based on user data input into the patient survey, inputting, by the user, one or more physiological parameters that allow remote monitoring of the patient, and comparing each of the one or more physiological parameters against a respective threshold and a respective change threshold.

In another aspect, the digital contact tracing through virtual medical information portfolio and on-ramp to testing includes a non-transitory computer readable medium having instructions stored thereon that, when executed by a processor, cause the processor to monitor health of a user, the instructions comprising receiving a text message or other electronic containing a QR code, bar code or other readable identifier that directs the user to a patient survey, categorize the user into one of a plurality of categories based on user data input into the patient survey, inputting, by the user, one or more physiological parameters that allow remote monitoring of the patient, and compare each of the one or more physiological parameters against a respective threshold and a respective change threshold.

It is to be understood that both the foregoing general description and the following detailed description are exemplary and explanatory and are intended to provide further explanation of the invention as claimed.

### BRIEF DESCRIPTION OF THE DRAWINGS

The accompanying drawings, which are included to provide a further understanding of the invention and are incorporated in and constitute a part of this specification, illustrate embodiments of the invention and together with the description serve to explain the principles of the invention.

FIG. 1 illustrates a computer-implemented flow diagram for infectious disease triage according to an example embodiment of the present invention.

FIG. 2 illustrates a computer-implemented flow diagram for using a contact tracing intake survey according to an example embodiment of the present invention.

FIG. 3 illustrates individual tracking and contact tracing according to an example embodiment of the present invention.

FIG. 4 illustrates a computer-implemented flow diagram for individual tracking and contact tracing according to another example embodiment of the present invention.

FIGS. 5A and 5B illustrate user-interfaces for inputting data into a Center for Disease Control (CDC) case survey for persons under investigation (PUI).

FIG. 6 illustrates a user-interface for visually tracking physiological parameters according to an example embodiment of the present invention.

FIG. 7 illustrates a login user-interface or patients, prescribers, and caregivers according to an example embodiment of the present invention.

### DETAILED DESCRIPTION OF THE INVENTION

Reference will now be made in detail to the embodiments of the present invention, examples of which are illustrated in the accompanying drawings. Wherever possible, like reference numbers will be used for like elements.

In some instances, the embodiments include user interfaces and associated methods for using a device. In some embodiments, the device is a portable communication device (e.g., a mobile phone or tablet). The user interface may include a touch screen, a gyroscopic or other acceleration device, and/or other input/output devices. In the discussion that follows, a portable communications device is used as an example embodiment. It should be understood, however, that the user interfaces and associated methods may be applied to other devices, such as personal computers and laptops, that may include one or more other physical user-interface devices, such as a keyboard and or mouse.

The portable communication device may support a variety of applications, such as telephone, text messenger, web enabled computer and calendar applications. The various applications that may be executed on the device may use at least one common physical user-interface device, such as a touch screen. One or more functions of the touch screen as well as corresponding information displayed on the device may be adjusted and/or varied from one application to another and/or within a respective application. In this way, a common physical architecture of the device may support a variety of applications with user interfaces that are intuitive and transparent. In the discussion that follows, a contact tracing application, such as Medication and Immunization Management Initiative (MIMI-Rx™), is used as an exemplary embodiment, but it should be understood that the user interfaces and associated methods may be applied to other applications. In addition, although the discussion that follows utilizes COVID-19 as an example of an infectious disease, the embodiments of the present invention are not so limited and may be readily applied to other infectious diseases.

The portable communication device and/or remote servers that implement the embodiments of the invention includes a bus or other communications mechanism for communicating information between components such as a processor, non-transitory memory, and external wired or wireless communication mechanisms.

Memory can be comprised of any combination of random access memory (“RAM”), read only memory (“ROM”), static storage such as a magnetic or optical disk, or any other type of machine or computer-readable medium. Communication device, such as a network interface card or other communications interface, to provide access to a network. As a result, a user may interface with the application and system directly or remotely through a network or any other method.

Memory can be a non-transitory computer-readable medium accessed by processor. A computer-readable medium may include both a volatile and nonvolatile medium, a removable and non-removable medium, a communication medium, and a storage medium. A communication medium may include computer readable instructions, data structures, program modules or other data in a modulated data signal such as a carrier wave or other transport mechanism, and may include any other form of information delivery medium known in the art. A storage medium may include RAM, flash memory, ROM, erasable programmable read-only memory (“EPROM”), electrically erasable programmable read-only memory (“EEPROM”), registers, hard disk, a removable disk, a compact disk read-only memory (“CD-ROM”), or any other form of storage medium known in the art.

Processor can also be operatively coupled via bus to a display, such as a Liquid Crystal Display (“LCD”) or touchscreen. Display can display information to the user. A keyboard and a cursor control device, such as a computer mouse, can also be operatively coupled to the bus to enable the user to interface with the MIMI-Rx™ application.

Memory can store software modules that may provide functionality when executed by the processor. The modules can include an operating system, modules for the MEVII-Rx™ application and system along with a suite of individual or multifactor authentication methods. In certain embodiments, MIMI-Rx™ can comprise a plurality of modules that each provide specific individual functionality for tracking user data. MIMI-Rx™ application can also be part of a larger system. Thus, the MEVII-Rx™ application can include one or more additional functional modules to include the additional functionality.

Processor can also be operatively coupled via bus to a database. Database can store data in an integrated collection of logically-related records or files. Database can be an operational database, an analytical database, a data warehouse, a distributed database, an end-user database, an external database, a navigational database, an in-memory database, a document-oriented database, a real-time database, a relational database, an object-oriented database, or any other database known in the art.

As will be described herein, the embodiments of the present invention are directed toward devices, systems, and methods for use of the MIMI-Rx™ application. MIMI-Rx™ provides a complete virtual health and telehealth product suite. The MEVII-Rx™ capability is a health and telehealth platform that is designed for the patient to own and control their data for life, from birth to death, at no cost to the patient. The technology incorporated into MIMI-Rx™ serves as both a means and a method for centralizing healthcare data and care. As described herein, the application can be configured for use as a scalable digital contact tracing platform to curb COVID-19 transmission as well as to triage non-COVID-19 upper and lower respiratory illnesses for early intervention and treatment.

With respect to contact tracing as it relates to COVID-19, each person/patient interview or documented COVID test or vaccination represents a point in time and without some element of persistent tracing the world will likely continue to experience multiple phases of the COVID pandemic of varying severity. Thus, some element of “persistent contact tracing” is urgently needed.

There are various traditional and manual ways to perform contact tracing. Each of these methods has an important role in reaching key populations and scaling up contact tracing both domestically and worldwide. Within the United States, four (4) major types of contact tracing are recognized and used in the administration of public health. These include: (i) in-person case-contact interviews, (ii) telephone-based case-contact interviews, (iii) digital/online-based case-contact interviews, and (iv) non-interview based tracing, likely using Bluetooth proximity sensing between phones.

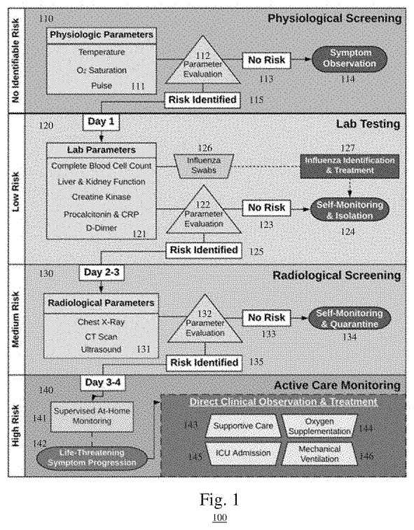

FIG. 1 illustrates a computer-implemented flow diagram 100 for infectious disease triage according to an example embodiment of the present invention. Flow diagram 100 is implemented by a combination of a user's portable electronic device and one or more remote servers that collectively implement the functionality of MIMI-Rx™.

As shown in FIG. 1, flow diagram 100 includes for stages corresponding to varying levels of risk: no identifiable risk stage 110 in which the user routinely undergoes physiological screening, low risk stage 120, a day or so later, in which the user undergoes lab testing, medium risk stage 130, at day 2 or 3, in which the user undergoes radiological screening, and high risk stage 140, at day 3 or 4, in which the user undergoes active care monitoring.

Turning to no identifiable risk stage 110 in which the user routinely undergoes physiological screening, the user may input into the application one or more physiologic parameters, at 111. For example, the user may input into the application temperature, O₂ saturation, and/or pulse. Other physiological parameters are also possible, such as weight, blood pressure, blood sugar, and a selection of other symptoms, such as headache or shortness of breath. Other clinical parameters and/or temporal parameters may be used. The user may input the one or more physiologic parameters one or more times per day. Increased frequency of input enables the application, or a remote healthcare provider, to better track trends in the physiological markers. A predetermined frequency of inputs may be preferred, such as 3-4 times per day, or that the individual has actually taken a vaccine or other treatment. Any person, including those in self-isolation or actual quarantine, may download the application and monitor and record symptoms as well as temperature and oxygen (O₂) saturation using a thermometer and a pulse oximeter every 8 hours or preferably at least 3 times per day. If the user forgets to enter the sensed physiological parameters, the application may transmit an alert to the user's portable electronic device reminding him/her to do so.

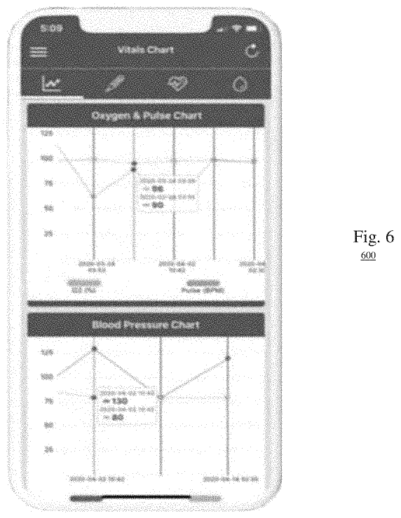

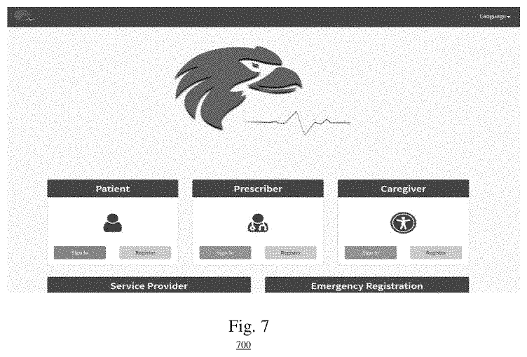

The application automatically calculates and graphically displays changes physiological parameters, such as temperature or O₂ saturation. For example, FIG. 6 illustrates a user-interface 600 for visually tracking physiological parameters according to an example embodiment of the present invention. The user also may share that information with a remote health care team at least once per day or more frequently if symptoms worsen. For example, FIG. 7 illustrates a login user-interface 700 for patients, prescribers, and caregivers according to an example embodiment of the present invention. User-interface 700 further includes login access for service providers, such as parameter laboratories, as well as hospitals and other emergency registration.

Returning to FIG. 1, and at 112, the application compares each of the inputted physiological parameters to a respective threshold. The storage and comparison of physiological parameters, other parameters, and data may be local to the user's portable device, at a remote server, and/or a combination thereof. Additionally, or alternatively, the application compares changes in each of the inputted physiological parameters to a respective change threshold. If the physiological parameters are within a normal range and/or no significant changes are detected, then parameter evaluation 112 determines that there is no risk 113 and the user may continue to periodically input physiological parameters and observe symptoms, at 114.

However, if any of the physiological parameters are not within a normal range and/or if significant changes to any of the physiological parameters are detected, then the application directs the user to low risk stage 120. For example, if the user experiences persistent temperature increase or persistent O₂ saturation decrease from that individual user's baseline values, the application directs the user to present for laboratory tests at a designated lab, at 121. Example laboratory tests may include complete blood count, coagulation profile, liver function test, kidney function test, procalcitonin, creatine kinase, C-Reactive Protein (CRP) and in some cases a blood glucose level and/or hemoglobin A1C. Additionally, or alternatively, nasal and oropharyngeal swabs 126 can be obtained for rapid diagnosis of all other more common respiratory pathogens such as influenza, respiratory syncytial virus, Bordetella pertussis infection (whooping cough), or Staphylococcus aureus infections of the nose and throat. At 127, early diagnosis of these more common infections and early treatment with appropriate therapy, such as Xofluza™ or TamiFlu™ as dosed in early onset of the influenzas A and B in outpatient settings is often more effective and decreases the likelihood of hospital-acquired infections if patients can be treated at home. These patients represent over 20% of the population presenting for symptomology and can be diagnosed through telehealth sessions, thus avoiding direct physical contact with care providers and care facilities.

At 122, the application retrieves lab parameters 121 from the designated lab and compares each of the lab parameters to a respective threshold. The MEVII-RX™ application may be communicative coupled with the designated lab to retrieve the user's lab results. Alternatively, an employee of the designated lab may input the lab results into the MIMI-RX™ system for viewing by the user and/or his/her healthcare provider. In yet another alternative, lab results received by the healthcare provider may be input into the MIMI-RX™ system.

If the lab parameters are within a normal range and/or no significant changes are detected, then parameter evaluation 122 determines that there is no additional risk 123 and the user may continue to periodically input physiological or clinical parameters and observe symptoms, at 124. Here, the user further may be instructed to self-isolate or quarantine. However, if any of the lab parameters are not within a normal range and/or if significant changes to any of the lab parameters are detected, then the application directs the user to medium risk stage 130.

At medium risk stage 130, the user is instructed to present to a designated diagnostic center for one or more radiological parameters 131. As symptoms do not improve or as laboratory findings are suggestive of a viral pneumonia, a chest CT scan, if available, chest x-ray or ultrasound may be ordered looking for bilateral ground glass opacities and interstitial markings that would then indicate the need for closer observation, rapid testing for COVID-19, and even possible hospital admission. At 132, the radiological parameters are evaluated at 132. Results of radiological parameters 131 can be evaluated using computer-aided diagnostics or by one or more healthcare professionals.

If the radiological parameters are present normally, then parameter evaluation 132 determines that there is no additional risk 133 and the user may continue to periodically input physiological parameters and observer symptoms, at 134. Here, the user further may be instructed to self-isolate or quarantine. However, if any of the radiologic parameters do not present as normal, then the application directs the user to high-risk stage 140.

At high-risk stage 140, the user's self-monitoring, in self-isolation or quarantine, is supervised by a remote healthcare professional, such as a physician or nurse. If life-threatening symptoms progress, the user is suggested to undergo direct clinical observation and treatment including one or more of supportive care 143, oxygen supplementation 144, intensive care unit (ICU) admission 145, and/or mechanical ventilation 146.

Critical to improving outcomes is early detection, persistent monitoring of clinical or vaccine regiments and early supportive care. Early detection coupled with self-isolation limits the potential risk exposure to the surrounding population, and assists to achieve pandemic control.

Persistently monitored person alert to a named contact of potential exposure and invite the individual to participate in the persistent digital daily check-in that prompts them to self-isolate, take temperature and oxygen saturation and report symptoms. The affected individual is invited to enroll into the MIMI-Rx™ application platform through the issuance of a QR-Code that documents the patient enrollment and starts the date and time for quarantine. This application is free to download, is designed for self-management of disease and is available to the individual for not less than 25 years. The application for persistent monitoring and/or testing (COVID-19 or other standards) allows the introduction of the sensors which today are currently covered by per member per month billing codes. Given the CMS 1135 Waiver, the provider that triggered the alert can also serve as the provider who writes the prescription for the per member per month (PMPM) order until more tests or vaccines become available and their accuracy and longevity of the vaccination efficacy is better validated.

If the patient's persistent monitors present an alert or the COVID-19 test comes back negative, the information is reported back to the named contacts and they are asked to continue monitoring for symptoms while maintaining appropriate levels of social distancing. If they develop symptoms which are suggestive of COVID-19, the system will once again remind them to self-isolate and help connect them with appropriate retesting. If the instituted vaccines develop indications that efficacy is not pertinent, additional persistent monitoring may be suggested until an additional vaccine or vaccine booster is received as treatment/prevention. These events also serve as the initiation/continuation of their own survey/interview as they are asked to report their own contacts.

If an individual's test comes back positive or their own monitoring alerts positive, the MIMI-Rx™ reports to them the result, and they are given a participant code to enroll in the contact surveillance survey themselves and subsequently identify their own contacts. Their identification of contacts then triggers the new individuals to receive their own QR-Code.

The thermometer and pulse oximeters, when coupled with phone tracking or wearable digital health sensors for children (FitBit, Oura Ring, Apple Watch, etc.) provide the best in class potential for symptom tracking of exposures and self-management of their own symptoms. If these data were shared by a user through the MEVII-Rx™ platform, this method would serve as the most accurate indicator of COVID-19 status in the history of all contact tracing while also serving for both the management of self-reported symptoms. This method of information capture and process for daily symptoms check-in will serve to affect patient adherence and education related to self-management of all chronic or episodic diseases.

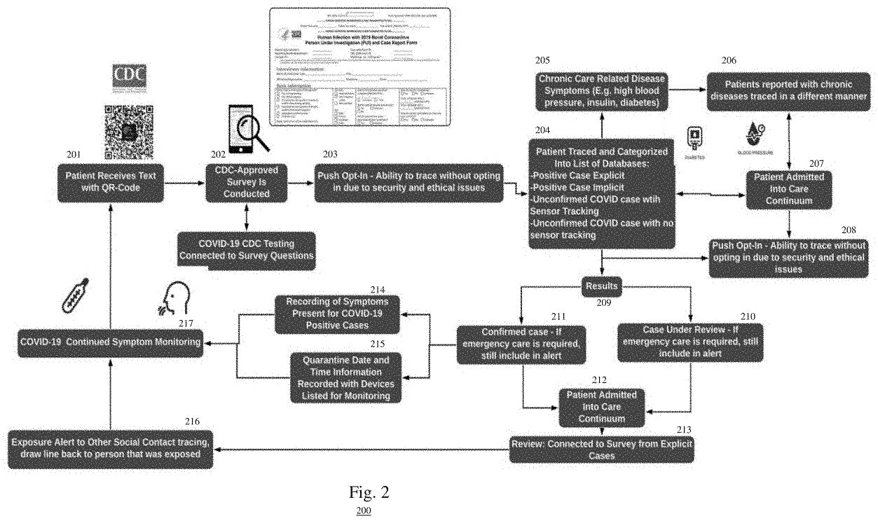

FIG. 2 illustrates a computer-implemented flow diagram 200 for using a contact tracing intake survey according to an example embodiment of the present invention. Flow diagram 200 is implemented by a combination of a user's portable electronic device and one or more remote servers that collectively implement the functionality of MIMI-Rx™.

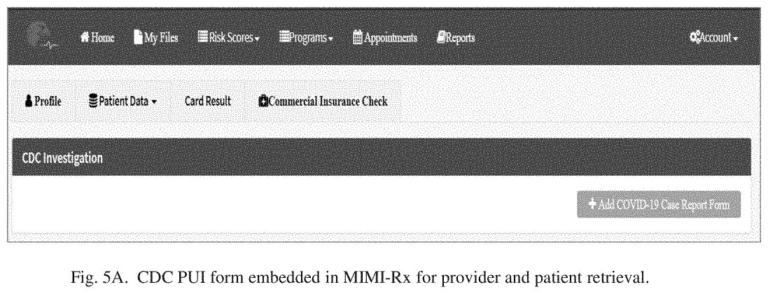

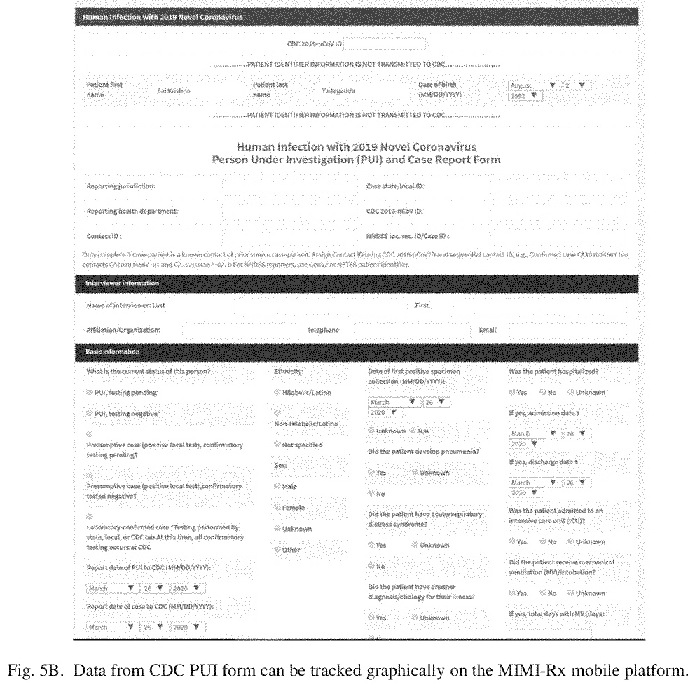

At the outset, the user receives a message, such as a text message, SMS message, or other electronic message which includes a digital unique identifier (e.g., QR code, bar code, etc.) that directs the user to the Center for Disease Control (CDC) case survey for persons under investigation (PUI), at 201. Here, during the clinical intake process or the survey interview, the MIMI-Rx™ system prompts the user (or a case worker, interviewer, or artificially-intelligent (AI) interviewer) also accessing to the application) to answer CDC case questions for persons under investigation regarding how the individual may have contracted the disease, at 202. For example, FIGS. 5A and 5B illustrate user-interfaces for inputting data into a CDC case survey for persons under investigation.

Returning to FIG. 2, the MEVII-Rx™ system prompts the user (or a case worker, interviewer, or AI interviewer) to provide information about other individuals with whom they have come in contact—their “contacts” during their 14-day infectious period, at 203. For example, has the user been to work, retail store, restaurant, cruise ship, airline, etc. In another example, if the user is a first responder (e.g., firefighter, police, etc.), has the user been in close proximity with colleagues or assisted people from the public. In the example where the user has been vaccinated, the time and date of the vaccination is utilized for the persistent monitoring where the efficacy of the vaccine is time orientated to diminished effectiveness. The user selects the ability to trace without opting in due to security and ethical concerns. This information allows the MIMI-RX™ system to map the contact items for review by the epidemiologist and to identify and monitor people most likely to develop infection and most likely to have their immunity reduced to the point of ineffectiveness. Recommendations for possible quarantine of these individuals ahead of testing and isolating them upon development of symptoms can be made while dynamically entering them into the triage process, as described in connection with FIG. 1, until a definitive positive or negative test result is available.

Next, the user is traced and categorized into one of a plurality of databases, at 204. The databases include: (i) positive case explicit, (ii) positive case implicit, (iii) unconfirmed COVID-19 case with sensor tracking (e.g., temperature, O₂ saturation, pulse, etc.), (iv) unconfirmed COVID-19 case without sensor tracking. Additionally, or alternatively, the user's characterization is correlated with the user's chronic disease which also may be tracked using MIMI-RX™, at 205. For example, the user may be undergoing care for a variety of chronic conditions, such as high blood pressure, diabetes, etc. At 206, users having one or more chronic conditions may be triaged and traced using additional parameters, such a blood pressure readings, blood sugar readings, etc. In addition, users having one or more chronic conditions may undergo additional levels of care on the care continuum by a healthcare provider, at 207. Critical to contact tracing and improving outcomes is early detection and early supportive care in the context of optimizing chronic conditions and medication adherence.

Users having one or more chronic conditions may pose increased risk of infections transmission. Again, the MIMI-Rx™ system prompts the user (or a case worker or interviewer coupled to the application) to provide information about other individuals with whom they have come in contact, such as healthcare workers, at 208.

At 209, MIMI-Rx™ determines if the user's symptoms are a confirmed case of COVID-19 infection, including those who have been vaccinated or a case under review. If the user is still designated as a case under review, and continues to be monitored for symptoms or date of last vaccination, and if emergency care is needed, the user receives an alert message, at 210. If the user has a confirmed case of COVID-19, and if emergency care is needed, including the requirement for a third or more vaccination, the user receives an alert message at 211. In either 210 or 211, users needing emergency care may undergo additional levels of care, on the care continuum, by a healthcare provider, at 212. Here, the user may be redesignated as a positive explicit case, at 213, and an exposure alert may be issued to determine if the user had contact with other individuals at 216 or just required the alert arriving due to the obsolesce of the previous vaccine.

Returning to a confirmed case at 211, the user is instructed to record symptoms present for COVID-19, at 214, and to provide quarantine date and time information, at 215. The host device of the MIMI-Rx™ application may assist in tracking quarantine dates, times, and locations using the date, time, and GPS location features of the device. Lastly, users having confirmed cases of COVID-19, other users having symptoms, or those whose vaccination is obsolete continue to persistently monitor symptoms over time at 217.

As noted above in connection with the completion of a CDC survey, at 202, the questions may be completed by either the user or a healthcare worker, preferably remote from the user. The intake process questions are automated into the MIMI-Rx™ technology and the interview information can be auto-populated into the CDC form. The completeness and quality of the contact data is mapped and the system algorithms are able to directly relate both explicit and implicit findings to estimate how successfully the system can be to curtail COVID-19 transmission. Therefore, the interviewer should be well versed and the process well designed to increase the validity of the responses and the timeliness of the reporting to potentially affected individuals. This area of epidemiological data analytics, alerts and reporting focuses on techniques for increasing the completeness and consistency of collection process and the process of indexing, classification and categorization of the questionnaire responses to include the means, methods and modality employed in reducing biases related to how interviewees recall or report information along with overlapping analysis in the reported population. One of the standard ways to improve questionnaire and clinical data collection quality is to form the baseline around known and trusted clinical information and administrative data, including date of last vaccination and to administer the questionnaire by standardized interview as a means of avoiding as much interviewer and interviewee bias as possible. Speaking with the interviewee helps to build trust and rapport, which may improve data completeness, however the differentiation of those contacts can eschew the data. Additionally, standardization of the questions and the process can drive the interviewer to guide questions or seek clarification, which may improve data accuracy. Confirmation through feed-back and feed-forward loops provide a constant verification of the validation process for tracing.

This process of reviewing known and trusted clinical and administrative information, interviewing persons affected, determining contacts, and monitoring the contacts serves as the standard contact tracing method, however the information used by Artificial Intelligence (AI) allows the ability to present plume analysis and scenarios based on additional features that do naturally serve as vectors towards predictable outcomes. For example: 1) when did an area present quarantine orders; 2) what is the population density; 3) what are the statistical baselines for population transportation; 4) when was the data of the last vaccination; 5) which manufacture medication did you receive, etc. The collection of the temporal and geospatial information may correlate time and location of individuals (e.g., user, and user contacts in nearby vicinity) to determine the risk of spread of COVID-19 or other infectious disease. Additionally, MIMI-Rx™ telehealth capabilities are extremely important in this process because the in-person interviews do put community healthcare workers and epidemiologists at risk for exposure and possible infection.

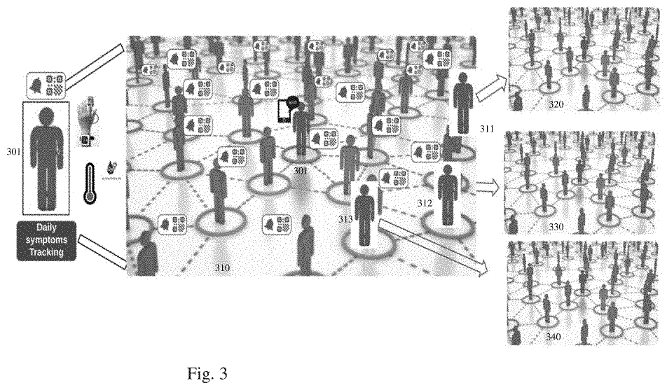

FIG. 3 illustrates individual tracking and contact tracing according to an example embodiment of the present invention. As shown in FIG. 3, a potentially infected person 301 may enter venue 310 having a plurality of non-infected people. As a consequence, some of the non-infected people who come into contact or close vicinity of potentially infected person 301 may become potentially infected, such as potentially infected persons 311, 312, and 313. As each of potentially infected persons 311, 312, and 313 enters a new venue, such as respective venues 320, 330, and 340 additional people become potentially infected. Example venues include retail spaces, restaurants, airplanes, cruise ships, classrooms, workplaces, etc. In some such venues, access to the space is tracked. By tracking entry/exit of the space, the venue owner can utilize the MIMI-Rx™ application and system to determine which individuals are likely to have come in close vicinity to a person at risk of infectious disease.

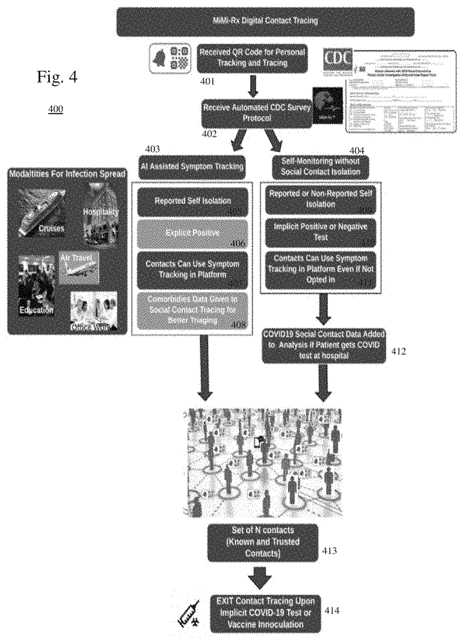

FIG. 4 illustrates a computer-implemented flow diagram 400 individual tracking and contact tracing according to another example embodiment of the present invention. Flow diagram 400 is implemented by a combination of a user's portable electronic device and one or more remote servers that collectively implement the functionality of MIMI-Rx™.

At the outset, the user receives a message, such as a text message, SMS message, or other electronic message, which includes a digital unique identifier (e.g., QR code, bar code, etc.) that directs the user to the Center for Disease Control (CDC) case survey for persons under investigation (PUI), at 401. Here, during the clinical intake process or the survey interview, the MIMI-Rx™ system prompts the user (or a case worker, interviewer, or AI interviewer also accessing to the application) to answer CDC case questions for persons under investigation regarding how the individual may have contracted the disease, at 402. For example, FIGS. 5A and 5B illustrate user-interfaces for inputting data into a CDC case survey for persons under investigation.

Returning to FIG. 4, the MIMI-Rx™ system prompts the user to select either AI assisted tracking at 403 or self-monitoring without social contact isolation at 404. If the user selects AI assisted tracking at 403, the user is prompted to report self-isolation or quarantine, at 405. Self-isolation or quarantine may continue for a predetermined period of time, or until the user obtains a negative COVID-19 test result. If the user receives a positive COVID-19 test result, at 406, contacts of the user can use the symptom tracking features of the MIMI-Rx™ application, at 407, and as described in connection with FIG. 1. Here, both the user and the users contacts comorbidity data, such as chronic illness is used for better monitoring and triage, at 408.

If the user selected self-monitoring without social contact isolation at 404, the user is instructed to report self-isolation or quarantine at 409. Subsequently, self-isolation or quarantine may continue for a predetermined period of time, or until the user obtains a negative COVID-19 test result. If the user receives a positive COVID-19 test result, at 410, contacts of the user can use the symptom tracking features of the MIMI-Rx™ application, at 411, and as described in connection with FIG. 1. In some instances, when the user gets a COVID-19, additional social contact data may be collected, at 412.

Here, contacts may be known. For example, one or more contacts may be identified by venue such as colleagues at work, passengers on an airplane, students in a classroom, patrons in adjacent hotel rooms, patrons at nearby restaurant tables, or passengers in nearby cabins in a cruise ship. Many such venues currently use RFID or other similar technology to secure entry to particular spaces. Entry and exit data may be correlated with data user COVID-19 exposure data to identify individuals who may pose additional risk for contracting COVID-19. Such individuals may be directed to use the symptom tracking features of the MEVII-Rx™ application, at 413 and as described in connection with FIG. 1. Individuals who obtain a negative COVID-19 test result or who have been vaccinated may exit the contact tracing feature, at 414.

The evidence-based systems and methods for data collection, limiting transmission, and utilization of known and trusted clinical information, as described herein, is designed to scale well within the context of the COVID-19 pandemic and the sheer number of persons that present within the respiratory transmission route. The systems and methods are designed to address the gaps presented by standard survey methods which are unable to scale to the vast degree of individual cases by extrapolating the analytical capabilities of the MIMI-Rx™ as a digital participatory contact tracing system by using AI and predictive analytical technology to function at scale and improve the data quality of self-reported sensor data, clinical reports and focused questionnaires.

The MEVII-Rx™ is already deployed nationally at no cost to patients or providers and statewide as the Maryland Covid-19 Command Center for 911 to 211 Maryland Responds Medical Reserve Corps (MRC) Caregiver alignment. The current collection process already employs technology that is designed to fill the role that a trained interviewer would normally have and the data collected throughout the intake process is consistent with the type and format of the data necessary in a standard epidemiological contact tracing operations. Specifically, the system is already focused for use in facilitating caregiver case management and tracking along with person and caregiver education both prior to and after a potential positive result is reported. This aspect will naturally improve completeness and accuracy of participant recall and reported responses while also presenting an avenue for persistent engagement with the affected individual and their caregiver and their contacts. These advantages in technological approach, data access and persistent engagement where a participant's recall are aided by temporal and geospatial time and location data are critical given the advantages of them all being traced, hosted and directly shared within the online platform with State Officials. The digitalizing of contact tracing also allows for the integration of personalized health technologies and deployment of clinical sensors (e.g., pulse oximeters and thermometers) to facilitate and augment the process of symptom tracking that will make this process the most advanced in the world. This methodology will serve as the only application of its type that has the capability to perform Bluetooth-enabled proximity sensing and symptom reporting to find contacts and trace them in a HIPAA compliant infrastructure which also serves as an integrated “Trace and Treat” framework. As it relates to contact tracing this method and system will enable assessment of the reliability for capturing contact events that result in disease transmission to serve as the ultimate check and balance as we focus on the control of disease and treatment of affected individuals.

When contact tracing is undertaken and enhanced with AI, persistent monitoring of diagnosed patients and those with chronic disease(s) these digital interviews will be empowering for the interviewee of both cohorts. By identifying individuals to whom the disease may have been transmitted, interviewees may utilize the MIMI-Rx™ application to host their medication, clinical and sensor data, they can directly engage in the process of limiting transmission, ensuring that other positive cases can be found earlier and isolated, hopefully before they transmit further. While discussions surrounding “flattening the curve” center on social distancing to prevent infection, the current methods represent the point in time and place without the ability to dynamically update from the use of persistent data. Monitoring of diagnosed and recovered patients and their contacts as well as those at high risk for severe illness based on co-occurring chronic disease through the use of persistently reporting devices serves to provide high quality information that can guide targeted attempts to limit transmission, early alerts to persons disproportionately susceptible to death, and a way to flatten the curve even for the infected individual. Furthermore, this provides the best opportunity for compromised persons to actively engage and successfully navigate the course of the disease.

The success of digital participatory contact tracing that relies solely on the contact's ability to access consistent and rapid diagnostic testing is unrealistic, thus persistent monitoring and reporting with AI analytics serves as the optimal modification to standard contact tracing methodologies. The rollout of a digitally-supported interview effort to find and isolate contacts is constructed in MIMI-Rx™ to work alongside the survey with its increased infrastructure designed to determine whether a contact is positive or negative while persistently receiving information to ensure the accuracy and relevance. Alerting contacts that they may have been exposed, without a mechanism for determining the result of that exposure, has caused undue angst and frustration, and will undermine both adherence to isolation and economic recovery. In addition to the importance of linkage to persistent monitoring, testing for protecting participant mental health assessments, linkage to testing, monitoring, training and alert infrastructure is critical to the general effectiveness of a targeted isolation strategy. Contact tracing in MIMI-Rx™ follows a cascading procedure, in which further interviews (e.g., digitally enabled by a remote interviewer or AI interviewer) and sensor alerts should be conducted to find contacts of contacts if a contact becomes a confirmed or presumptive positive case. Venues using the application and system also may identify contacts. Without the ability to either test or monitor through sensors for reported exposed contacts or indications of exposure, the cascading nature of this system will break, and continued transmission that goes undetected will likely occur. The MIMI-Rx™ screens (e.g., FIG. 6) represent both the sensor collections for temperature and oxygen saturation along with the CDC survey (e.g., FIG. 5B) as they are designed to work hand in hand.

Until universal testing is available, it is imperative that certain industries in the U.S. and globally that depend on transportation (airlines, trains, cruise ships, commercial maritime trade, etc.), outdoor-focused tourism, hotels, restaurants, gaming and other critical infrastructure industries, utilize MEVII-Rx™ have a mechanisms to reduce the risk posed by infectious disease. For example, the MIMI-Rx™ application may be configured to generate a digital health identifier that may be presented by the user at such venues to indicate that the user is low risk for infectious disease. Here, the digital health identifier may include an aggregation or automated evaluation of the user's health-related parameters to present a low, medium, or high risk indicator, for example.

The MEVII-Rx™ application and system have been configured from an initial focus on 11,000,000 persons on Medicare affected by home health services to every American to scale to include all components of the transportation and critical infrastructure services as reopening of the national and international economies is on the horizon. This AI-enhanced technology and its integrated survey expands the contact tracing from service only in areas with sufficient testing infrastructure to support the testing load, to all areas and segments of society while maintaining privacy and HIPAA compliance. The system can be deployed in close partnership with public health agencies at the federal level (HHS-Office on Aging), State (MRC Command Center in Maryland), Partnership with the Association of State and Territorial Health Officials (ASTHO) and Local (Morehouse School of Medicine, Outpatient Clinics and numerous Area Offices on Aging). Close partnership with local public health agencies is preferred to expand deployment of MIMI-Rx™. This approach to persistent monitoring and survey-based digital contact tracing will work hand-in-hand with telehealth interviews conducted by public health officials, providers and deployed sensors focused at persistent monitoring and early alerts.

Connection to COVID-19 testing is critical for all phases of individual health and persistent syndromic surveillance. Though current national models are designed to inform individuals of exposure, it is not ethical (or expeditious) to tell an individual “they may have been exposed to COVID” without the ability to get them tested. It is clear both nationally and internationally that the threshold for alerting an exposure should be tied to testing capacity, however with the lack of an ability to test, it has become clear that the use of probability alerts based on known and trusted data and laboratory reports serves as both an accurate and early alert to the contact tracing methods.

This disclosure is not alone in its effort to define new models in an attempt to curb the COVID-19 pandemic. Other digital contact tools and technology are designed to electronically track the person with unclear definitions of data management and control. Many of these have focused on Bluetooth-enabled proximity tracking (e.g., TraceTogether, COVID Watch, NOVID, COVID Link, PEPP-PT, etc.).

MIMI-Rx™ is a digital health platform that recognizes the importance of patient self-management of disease, use of clinical decision support by AI-focused functions, utilization of the Internet of Health Things (IoHT), all working in conjunction with the model of traditional, epidemiological contact tracing and interviews. Use of digital tools to enhance data collection, increase interaction with the named contacts, and importantly, allows for widespread use and scaling of implementation.

The embodiments of the invention, such as the MIMI-Rx′ application and system, provide a secure, real-time, interoperable analytics package that uniquely stratifies an individual's risk, providing assessment of clinical tests, screenings, and pre-hospital, forward triage capability. Further, early diagnosis and treatment of respiratory illnesses that are not COVID-19 and early management of exacerbations of chronic medical conditions will preserve scarce resources, improve the health of the community, and protect the healthcare workforce.

The four (4) types of contact tracing may be implemented and used in parallel and the MIMI-Rx™ platform serves the only nationally scaled infrastructure that has served internationally within the North Atlantic Treaty Agency (NATO), certified to adhere the health care Standardization Agreement 2517 (STANAG 2517) and subsequently deployed throughout the United States, directly affecting the Medications and Immunizations of over 325,000,000 Americans. MIMI-Rx™ enables patients to own control and access of their health data with the ability to harvest and maintain data from disparate systems to communicate with their health care team. Contact tracing requires that as a society we know who has been followed up on and how current and accurate that information is as reported. This disclosure presents the next generation contact tracing that is scaled for secure national and international support with full patient control of their data.

It will be apparent to those skilled in the art that various modifications and variations can be made in the digital contact tracing through virtual medical information portfolio and on-ramp to testing of the present invention without departing from the spirit or scope of the invention. Thus, it is intended that the present invention cover the modifications and variations of this invention provided they come within the scope of the appended claims and their equivalents.

What is claimed is:

1. A computer-implemented method for monitoring health of a user, the method comprising:
receiving a text message containing a digital unique identifier that directs the user to a patient survey;
categorizing the user into one of a plurality of categories based on user data input into the patient survey;
inputting, by the user, one or more physiological parameters that allow remote monitoring of the patient; and
comparing each of the one or more physiological parameters against a respective threshold and a respective change threshold.

2. The computer implemented method according to claim 1, wherein the physiological parameters include one or more of temperature, oxygen saturation, and pulse rate.

3. The computer implemented method according to claim 1, further comprising receiving one or more lab parameters in response to the comparison of the one or more physiological parameters against a respective threshold and a respective change threshold.

4. The computer implemented method according to claim 3, further comprising receiving one or more radiological parameters in response to the comparison of the one or more lab parameters against respective thresholds.

5. The computer implemented method according to claim 1, further comprising sending an alert to one or more contacts of the user to indicate potential exposure to an infectious disease.

6. The computer implemented method according to claim 1, wherein the one or more contacts are identified as having been in close vicinity as the user.

7. The computer implemented method according to claim 6, wherein each of the one or more contacts are requested to input one or more physiological parameters to allow remote monitoring.

8. A portable electronic device for monitoring health of a user, the portable electronic device comprising:
a processor;
a storage memory coupled to the processor, the processor configured to:
receiving a text message containing a digital unique identifier that directs the user to a patient survey;
categorizing the user into one of a plurality of categories based on user data input into the patient survey;
inputting, by the user, one or more physiological parameters that allow remote monitoring of the patient; and
comparing each of the one or more physiological parameters against a respective threshold and a respective change threshold

9. The portable electronic device according to claim 8, wherein the physiological parameters include one or more of temperature, oxygen saturation, and pulse rate.

10. The portable electronic device according to claim 8, further comprising receiving one or more lab parameters in response to the comparison of the one or more physiological parameters against a respective threshold and a respective change threshold.

11. The portable electronic device according to claim 10, further comprising receiving one or more radiological parameters in response to the comparison of the one or more lab parameters against respective thresholds.

12. The portable electronic device according to claim 8, further comprising sending an alert to one or more contacts of the user to indicate potential exposure to an infectious disease.

13. The portable electronic device according to claim 8, wherein the one or more contacts are identified as having been in close vicinity as the user.

14. The portable electronic device according to claim 12, wherein each of the one or more contacts are requested to input one or more physiological parameters to allow remote monitoring.

15. A non-transitory computer readable medium having instructions stored thereon that, when executed by a processor, cause the processor to monitor health of a user, the instructions comprising:
receiving a text message containing a digital unique identifier that directs the user to a patient survey;
categorizing the user into one of a plurality of categories based on user data input into the patient survey;
inputting, by the user, one or more physiological parameters that allow remote monitoring of the patient; and
comparing each of the one or more physiological parameters against a respective threshold and a respective change threshold.

16. The non-transitory computer readable medium according to claim 15, wherein the physiological parameters include one or more of temperature, oxygen saturation, and pulse rate.

17. The non-transitory computer readable medium according to claim 16, further comprising receiving one or more lab parameters in response to the comparison of the one or more physiological parameters against a respective threshold and a respective change threshold.

18. The non-transitory computer readable medium according to claim 17, further comprising receiving one or more radiological parameters in response to the comparison of the one or more lab parameters against respective thresholds.

19. The non-transitory computer readable medium according to claim 15, further comprising sending an alert to one or more contacts of the user to indicate potential exposure to an infectious disease.

20. The non-transitory computer readable medium according to claim 8, wherein the one or more contacts are identified as having been in close vicinity as the user.
